(19) United States
(12) Patent Application Publication
(10) Pub. No.: US 2011/0152696 A1
(43) Pub. Date: Jun. 23, 2011

(54) AUDIBLE BIOFEEDBACK HEART RATE MONITOR WITH VIRTUAL COACH

(75) Inventor: Richard H. Ryan, Sherman Oaks, CA (US)

(73) Assignee: Hall Ryan Laboratories, Inc., Sylmar, CA (US)

(21) Appl. No.: 12/975,263

(22) Filed: Dec. 21, 2010

Related U.S. Application Data
(60) Provisional application No. 61/289,251, filed on Dec. 22, 2009.

(51) Int. Cl. G09B 19/00; A61B 5/024

(52) U.S. Cl. 600/481; 434/247

(57) ABSTRACT

A system and associated method that provides a virtual coach to a person during an exercise routine includes a sensor used to sense the level of a physiological parameter of the exerciser, such as heart rate, a processor programmed to compare the sensed physiological parameter to a target level of the physiological parameter, and, depending on the comparison, provide coaching prompts to the exerciser. Coaching prompts include audible and visual prompts. In another feature, a visual image of a virtual coach may be made available for display to the exerciser, and in yet another feature, a video of the virtual coach saying the audio prompt may be displayed. The system allows for selection from a plurality of virtual coaches. The audio and video of virtual coaches are recordings of real people and may be produced or obtained from other sources. In one embodiment, audio, visual, and video of the virtual coach may be downloaded from the Internet.

### CROSS-REFERENCES TO RELATED APPLICATIONS

This application claims the benefit of U.S. application Ser. No. 61/289,251, filed Dec. 22, 2009, incorporated herein by reference in its entirety.

### BACKGROUND

The invention relates to a system and associated method for informing a person who is exercising of the state of his or her physical condition, and more particularly, to a system and method that provide a person with sensory feedback in the form of virtual coaching based on a monitored physiological parameter.

Many people who exercise strive to maintain certain performance goals or improve their performance. A runner or bicyclist, for example, may desire to improve performance by increasing speed over a defined distance or by increasing total distance that can be traveled without resting. One well-known method that exercisers use to improve performance is to set, and strive to reach, performance goals. In addition to this, there are people who are directed by their doctor to achieve a certain level of cardiovascular exercise, which is challenging when it is required of a person who is not normally self-motivated to exercise. All of this requires that the exerciser measure performance during the training session and compare the measured performance to the pre-defined goals. For example, if an exerciser desires to increase his or her average running speed, he or she would set an average speed goal, measure an actual average speed during a training session, and then compare the average speed goal with the actual average speed. In this manner, the exerciser is provided with performance feedback that can be used as a guide for future training sessions.

To make measurements of actual average speed or overall speed during the exercise period, an exerciser typically measures an elapsed time of the training session using a stopwatch, measures the distance traveled during the session using a map or landmarks, and divides the distance traveled by the elapsed time. Unfortunately, this requires that the exerciser carry the stopwatch and remember to start and stop the stopwatch at appropriate points during the training session. It also requires that the exerciser determine the distance traveled. If the exerciser uses a non-standard or arbitrarily defined course, the distance can sometimes be roughly determined from a vehicle's odometer, though the result may be significantly inaccurate. Where the course cannot be driven with a vehicle, such as, for example, where the course is over open country or over water, some other means of determining distance must be devised. Pedometers, for example, are known in the prior art for measuring distance traveled. Unfortunately, pedometers suffer from a number of disadvantages, including, for example, that they must be properly calibrated; are inaccurate for most forms of movement other than running; and the runner must maintain consistent strides regardless of such factors as changing terrain. Following an arbitrary course can also make repeating the course extremely difficult because the exerciser may not recall the course's exact route. Furthermore, this method makes it very difficult for the user to be aware of performance and progress toward reaching the pre-defined goals during the training session.

As is apparent, the above concerns may adversely affect the ability to obtain clear feedback on the attainment of improvement in an exercise program. In many cases, due to the complications and complexities expressed above, people simply exercise for a predetermined period of time, such as thirty minutes. Also, in some cases, certain individuals are ordered to exercise by their physician but at the same time are ordered to not exceed a certain heart rate during that exercise. People suffering from certain coronary conditions, vascular disorders, such as electrical or mechanical disturbances of the heart, or aneurysms, must be careful about generating excessive blood pressure. Limiting the heart rate to a certain maximum has been found to be useful in controlling the blood pressure. The objective is to raise the heart rate to a level that results in good cardio-vascular exercise for a significant period yet not exceed a certain heart rate. The difficulty arises in determining if the exerciser has achieved the desired heart rate, but has not exceeded it. In these cases, a physiological condition or parameter of the exerciser needs to be monitored, as opposed to an athletic performance parameter.

Portable monitors for measuring heart rate exist but typically these devices report information to a user in a digital numerical format by means of a screen attached to the device itself. Thus, the user must look at a device on his or her wrist or elsewhere, while he or she is running, walking, or otherwise exercising in order to take note of heart rate. Where looking at a monitor to see heart rate requires altering or perhaps interfering with the particular exercise or other activity, the exercise may be negatively affected or the person may not adequately check the monitor.

Many present systems track only the performance of an exerciser; e.g., distance run, elapsed time of exercise, speed of exerciser, repetitions completed, lap time, and others. However, as in the paragraph above, tracking an actual physiological parameter of an exerciser, such as heart rate, can be more important and required for some.

In the case of many exercisers, there exists a very low motivation factor that can interfere in the successful attainment of exercise goals. In the case of runners, for example, where the exercise is repetitive over the entire distance and time duration, motivation to complete the course or the duration may be lacking, and in a significant percentage of people, the exerciser may not even try to begin the exercise due to expected boredom. Many people are not exercising for competitive purposes; they may only exercise for health reasons and the boredom factor may cause them to forgo needed exercise.

To counteract boredom or other interfering factors that tend to de-motivate people from exercising, many exercisers resort to the distraction of an entertainment source during their physical exercises. Listening to music, news broadcasts, discussions, and in the case of a stationary exercise such as a treadmill or stationary bicycle, television, has been found to aid many in keeping up their physical exercise regimens. However, in many cases, this is still not enough.

It has also been found that even better motivation to complete an exercise routine, and improve over time, can result from the use of a coach. Many people employ personal coaches, also called personal trainers, to motivate them and encourage them to accomplish their exercise routines. However, such coaches are not available to a substantial number of exercisers, due to monetary restrictions, unavailability, or other reasons. But a need and desire still exist in many people for the benefits of a coach. Coaches not only provide encouragement and advice and in some cases scolding, but also provide interim feedback. For example, during a run, a coach may time the runner on each lap, or during segments, and report to the runner at such intervals that he or she is attaining the goal or needs to change something to attain the goal. The coach also may use words of encouragement at this time, such as “Good effort” or harsher language, such as “Try harder, you can do it with more effort.” This interim feedback and encouragement has been found to have a very positive effect on many exercisers and consequently, goals are met and improvement can result.

Hence, a need has been recognized for a system and method to monitor a selected physiological parameter of an exerciser and provide more convenient and less intrusive interim sensory feedback to the exerciser to motivate and encourage the exerciser to continue or to alter his or her activity so that a goal may be attained. A need has also been recognized for a coach to provide feedback during an exercise routine. The present invention fulfills these needs and others.

### SUMMARY OF THE INVENTION

Briefly and in general terms, the invention is directed to a system and associated method for providing a virtual coach to an exerciser. More particularly, there is provided a system for monitoring a physiological parameter and coaching an exerciser during exercise comprising a sensor configured to sense a physiological parameter and provide a sensor signal representative of the actual level of the sensed parameter, an input device configured to provide a target level of the physiological parameter, a processor programmed to receive and compare the target level of the physiological parameter with the actual sensed level and in response to the comparison, play an audible coaching prompt indicative of the difference between the actual and the target, and an audio transducer configured to receive the played audible coaching prompt and transduce it into audio energy for listening by the exerciser.

In further more detailed aspects, the physiological parameter sensed comprises heart rate. Additional coaching prompts are used that vary in intensity of coaching. At least one prompt comprises a recording of a voice message from an actual person. Wherein the processor is further programmed to select a prompt based on whether the actual sensed parameter is higher or lower than the target rate.

Further more detailed aspects include the system comprising a memory, wherein the processor is further programmed to store in the memory the last prompt provided and in the event that a subsequent prompt is needed, the processor is further programmed to refer to the stored prompt in selecting the next prompt. The memory has stored a plurality of prerecorded audio prompts from which the processor is programmed to select in playing an audio coaching prompt, the stored prompts having a predetermined sequence for playing, the processor being programmed so as to retrieve and play the prompts in the order of that predetermined sequence. The system further comprises a display, wherein the memory has stored a visual image of a coach face, and wherein the processor is further programmed to retrieve that visual image from the memory and output that image to the display when playing an audio prompt.

Yet additional more detailed aspects include the memory having stored a video of a coach providing the audio prompt played by the processor, the processor being further programmed to retrieve that video from the memory and output that image to the display when playing an audio prompt. Further, the system also comprises an input port; wherein the processor is further programmed to receive a live video prompt at the input port, record the live prompt as a video file, and store that recorded video file in the memory for playing as a prompt. Additionally, the processor is further programmed to receive a prerecorded video prompt file at the input port and store that received video file in the memory for playing as a prompt.

In regard to the associated method, there is provided a method of monitoring a physiological parameter and coaching an exerciser during exercise, comprising measuring a physiological parameter of an exerciser, selecting a target level for the physiological parameter of an exerciser, comparing the measured physiological parameter to the target level, outputting a coaching audio prompt to an exerciser based on the difference between the measured physiological parameter and the target physiological parameter, and in the event that a visual image or a video of a coach saying an audio prompt is desired, outputting a visual image and/or a video of a coach to an exerciser.

### BRIEF DESCRIPTION OF THE DRAWINGS

FIG. 1 is a diagrammatic view of an exerciser in motion using an automated sensory feedback system and method in accordance with aspects of the invention, that provide a virtual coach, the coaching being based on a defined goal and a sensed physiologic parameter of the exerciser. The figure showing alternative physiological sensors, an entertainment source and virtual coach, and an output in the form of earphones;

FIG. 2 is a rudimentary flowchart of a portion of a virtual coach method in accordance with aspects of the invention, in particular, showing selected coaching prompts as one embodiment;

FIG. 3 is a more detailed flow chart of a physical monitoring and virtual coach method in accordance with aspects of the invention showing the output signal of a sensor, the processing of that sensor signal, and in response, the delivery of coaching prompts that are played to the exerciser appropriate to the exerciser's physical condition and history in the exercise routine undertaken as compared to the predetermined exercise regimen;

FIG. 4 is a schematic block diagram of a sensory feedback and coaching system in accordance with aspects of the invention including audible and visual feedback to an exerciser of the heart rate of the exerciser at preselected intervals, and further showing the feature of obtaining audio and visual prompt files from an external source to augment the coaching feature, the system further providing priority of coaching feedback over the entertainment source automatic control of the audio levels in accordance therewith;

FIG. 5 is a diagram of forms of visual images of virtual coaches, in accordance with another aspect of the invention;

FIG. 6 is a diagram of a handheld device on which is shown the video image of a virtual coach with heart rate and other data, the coaching prompts being received audibly through the connected earphones; and

FIG. 7 is a diagram of a system in accordance with aspects of the invention in which coaching prompts in the form of audio files, video files, and image files may be uploaded to a world wide web commercial data base server from a talent production source, and those files may be previewed, selected, paid for, and downloaded to an exerciser's computer and device for playing, and in another aspect shown, the audio files may be made personally at the exerciser's computer for download to the exerciser's device for playing.

### DETAILED DESCRIPTION OF PREFERRED EMBODIMENTS

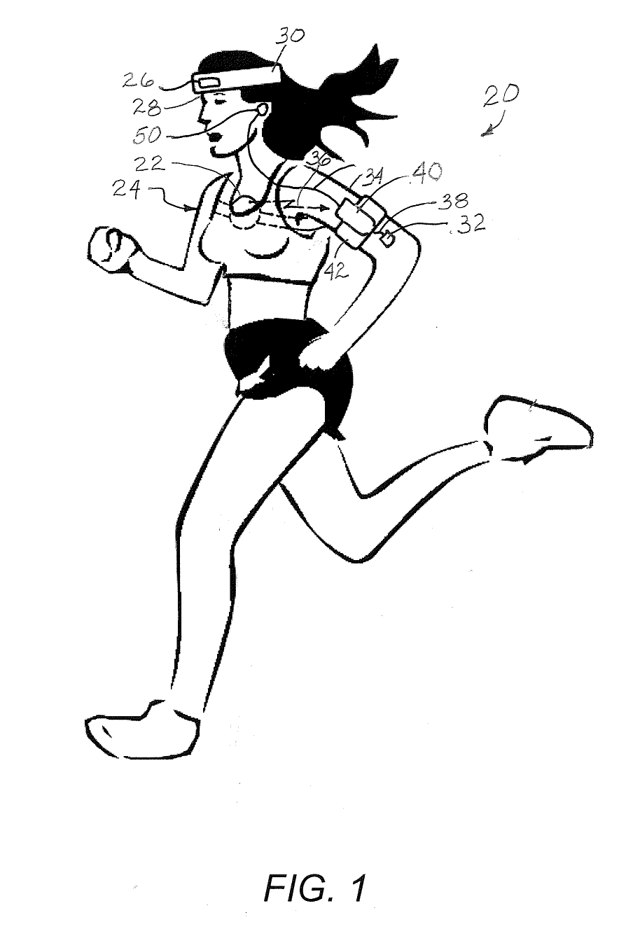

Referring now in detail to the drawings, in which like reference numerals refer to like or similar elements or steps among the views, there is shown in FIG. 1 an audible biofeedback heart rate monitor with virtual coach system 20 having a heart rate sensor 22 that is removably attached to the chest of an exerciser 24. Also shown are alternative embodiments of sensors, in which a sensor 26 may be attached to the forehead 28 of the user (through a sweat band 30 or other device) or in yet a further embodiment, a heart rate sensor 32 may be adhesively or otherwise attached to a limb of the user, such as the upper arm 34, as shown.

Sensors such as those depicted employ known means for sensing a user's heart beat, or other physiological parameter, and determining the heart rate, and are configured with known means to convert the heart rate into an electrical signal that is transmissible to a processor worn on the body of the user or located elsewhere. Such known means for sensing the heart beat may rely on laser, electric pulse, piezoelectric principles, or other known means to detect the heart beat.

The heart rate sensor 22 electrical signal output may be transmitted wirelessly by radio signal 36 (as shown for the case of the chest sensor 22), or may be transmitted by wired connection 38 to a processor 40 as shown in regard to the upper arm sensor 32. In FIG. 1, the wireless sensor signal 36 is transmitted to the processor 40 attached to the exerciser 24 by an armband 42 positioned on the upper arm 34 in the embodiment shown. Earphones 50 are connected to the processor 40 by wired connection 52 in FIG. 1 to provide the exerciser 24 with audible coaching prompts, as discussed below.

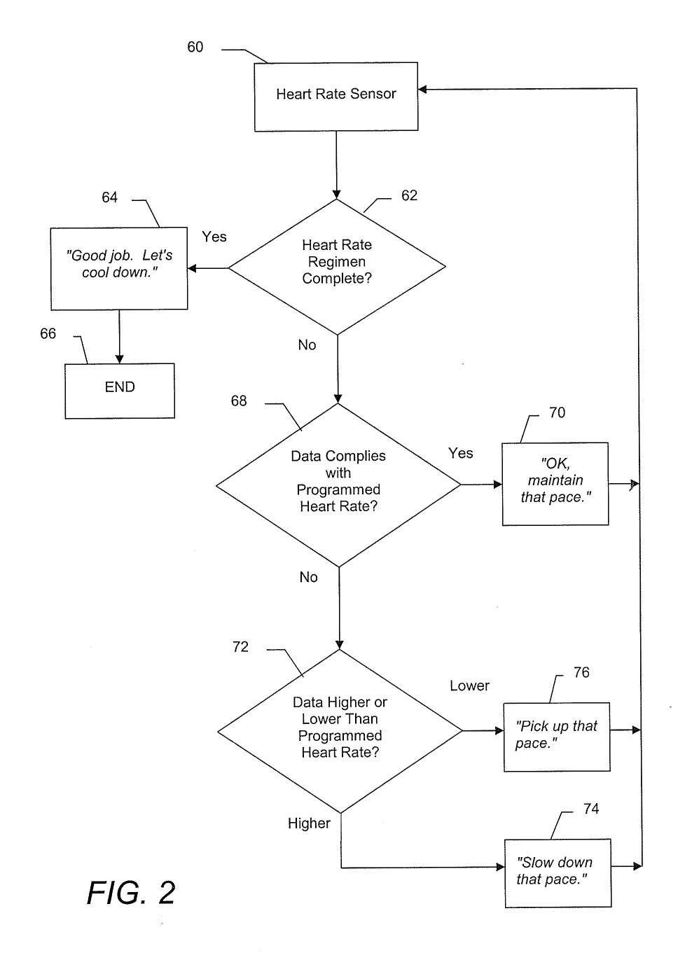

In accordance with aspects of the invention, the sensor signal 36, which represents the actual heart rate Ra of the exerciser 24, is compared against a target heart rate Rt. Depending on the results of the comparison, coaching is provided to the exerciser. Turning now to FIG. 2 in conjunction with FIG. 1, it is shown that the output of a sensor 60 is processed in accordance with the method embodiment shown. The processor is programmed to determine if the heart rate regimen of the user 22 is complete 62. That is, the mileage has been run, or the time period for exercising has expired, or the exerciser's heart rate has been elevated to a certain level for a prescribed period of time, or other exercising factor input by the exerciser has been accomplished. If the exerciser's regimen has been competed, the processor will provide a prompt or message notifying the exerciser of this, such as “Good job. Let's cool down” 64, or other, and may include a visual prompt also, depending on the embodiment. The regimen may then end 66, or in other embodiments, further processing and possibly data communications to a remote server or other data base may occur about the exerciser's accomplishments.

If the exerciser's heart rate regimen is not yet complete, the processor is programmed to now determine if the heart rate sensor data indicates exerciser compliance with a programmed heart rate 68. The processor is programmed to compare the data representing the actual heart rate (Ra) to the target heart rate (Rt) as was programmed by the exerciser. If the comparison indicates that the Ra is at or within a certain tolerance band of Rt, the processor will provide an audible and/or visual prompt indicating the results of this comparison 70. The method then returns to the heart rate sensor data output step 60.

In the event that the actual heart rate Ra does not comply with the programmed heart rate Rt, the processor is further programmed to make a determination as to whether the actual rate is higher or lower than the programmed rate 72. If the Ra is higher than the Rt, the processor provides a prompt 74 recommending action to remedy the difference in rates, such as “Slow down that pace.” If the Ra is lower than the Rt, the processor is programmed to also provide a prompt 76 to remedy the difference in rates, such as “Pick up that pace.” After providing either one of the foregoing prompts, the method returns to the step of monitoring the heart rate data of the exerciser 60.

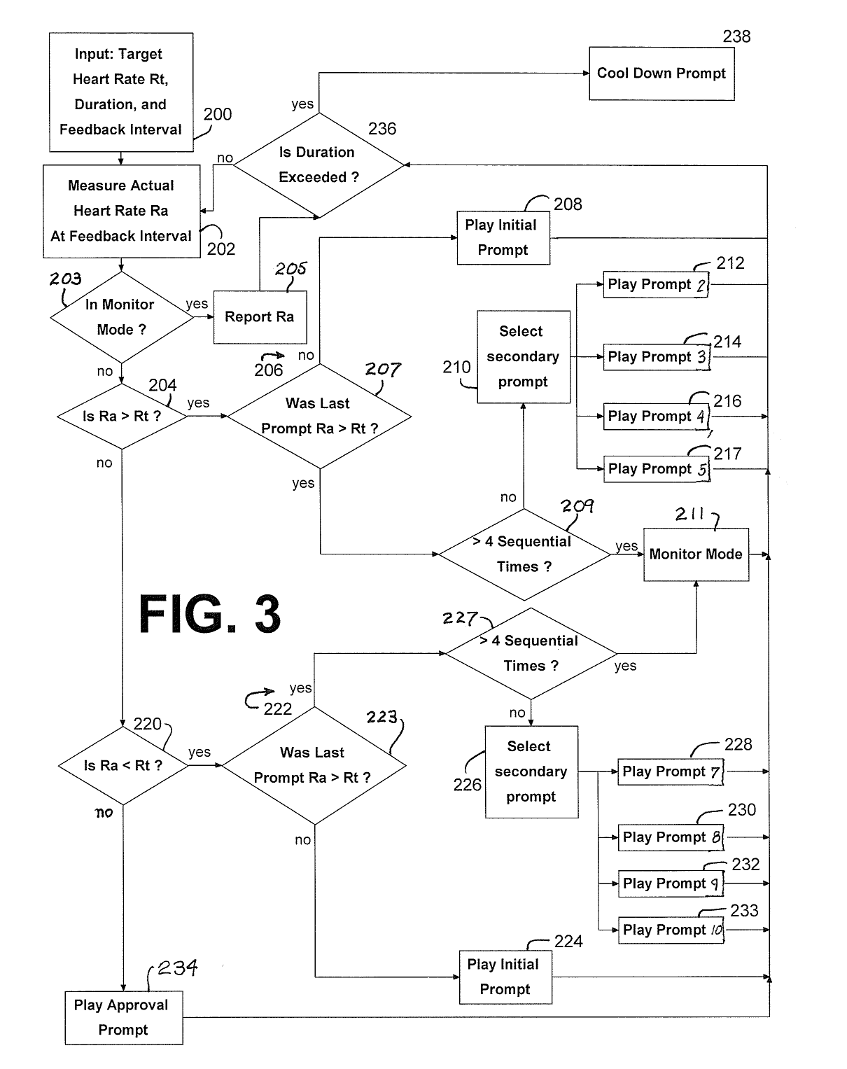

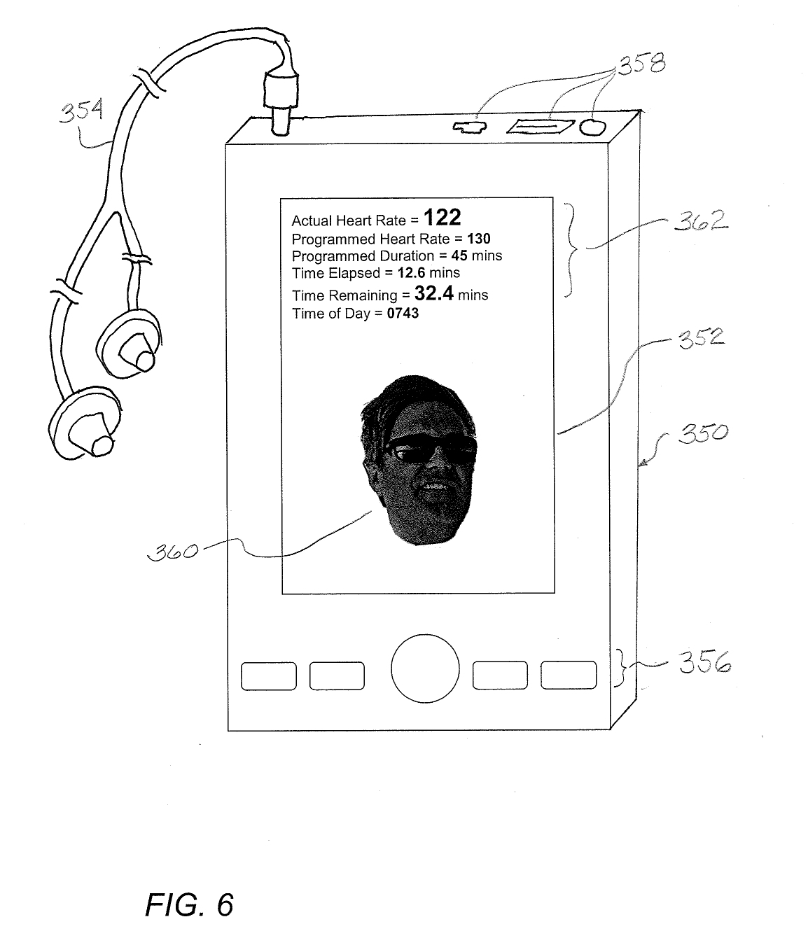

Turning now to FIG. 3, there is shown a flow chart of data processing in accordance with more detailed method aspects of the invention. As an initial step 200, the target heart rate Rt is input, as well as the duration of the exercise routine, and the length of the feedback interval. The feedback interval is time at which the processor provides coaching feedback to the exerciser. For example, if an interval of thirty seconds is selected, then at every thirty-second interval, the processor will provide a coaching prompt. A one-minute or other time-based interval may be selected or entered, or the user may select an occurrence of an event that is not time based, at which a prompt will be given. For example, the user may request a prompt every thirty seconds until the target heart rate has first been reached, and then prompts will not be given unless the user's heart rate varies from the target by more than a predetermined percentage, such as twenty-five percent. Entry of these parameters may be performed by known data entry means such as, for example, a visible data screen in combination with a set of buttons or keys with numerical values (see FIG. 6), and/or a toggle switch in combination with a cursor, or by touch screen technology, or by other means.

In another embodiment, the target heart rate may be programmed to vary over the course of an exercise routine. For example, the user may enter a rapid start to reach Rt followed by a lengthy cool down period, or may enter a lengthy ascent to the target Rt, followed by a rapid cool down period. Variations are within the scope of the invention. Another important parameter that must be entered is the duration of the exercise routine (step 200). For example, the user may wish to perform a certain routine that lasts for only twenty minutes. This time duration is entered along with the target heart rate. The processor is configured with a time clock that enables it to measure the time taken from the start of the routine, and report to the user how far the routine is from completion of the target time, as provided in one embodiment. In another case, the user may want to run for twenty minutes after the target heart rate has been reached, even if it takes ten minutes to reach it. Indeed, it is within the scope of the present invention that a series of specific discrete exercise routines may be entered into the processor 12. These routines may call out a variation in the exertion required over a period of time, and all that remains for the exerciser is to enter his target heart rate Rt which will take into account his personal fitness level and general physical condition.

Once these parameters are entered 200 and stored in the processor 40, the actual heart rate of the exerciser is measured 202 at a certain interval of time. The actual heart rate Ra is determined from the signal 36 coming from the heart rate sensor 22 (see FIG. 1). The processor determines if the system is in the “monitor mode” in step 203 (described below). If so, the actual heart rate is simply reported 205. The processor 40 then compares Ra with Rt (step 204), and if Ra is greater than Rt, the processor performs a series of steps in a subroutine 206 designed to warn the user that he or she is overexerting himself or herself and should slow down. However, another feature of the present embodiment is to monitor previous prompts provided to the exerciser. For example, the subroutine 206 first checks to see if the last coaching prompt was also based on a determination that Ra was greater than Rt 207. If not, the audio device selects and plays an “initial prompt” audio file (step 208), which would merely give the exerciser a first warning that he or she is overexerting. An example prompt may be “Slow down, your heart rate exceeds your selected maximum.”

However, if the last prompt output was based on a determination that Ra>Rt, then it means that the user is persisting in exceeding his or her target heart rate Rt, and the processor selects 210 a secondary prompt file 212-217 and plays the selected prompt file to warn him/her about possible overexertion. For example, the prompt may say, “You are continuing to overexert yourself. Please slow down.” A series 214 to 217 of selectable prompts having increasing intensity of coaching is provided. In conjunction with the embodiment above, these intensified prompts occur for a pre-programmed number of times 209 before the system assumes the exerciser is no longer responding to prompts and does not intend to maintain the pre-programmed regimen, at which point, the audible feedback would state something such as “You're no longer listening to my coaching advice, so I'm going to just monitor your heart rate.” The system and method then switch to “monitor mode” 211 as described above, during which the processor simply reports the measured heart rate at an interval. In one embodiment, all measurements of the heart rate and comparisons of the actual heart rate to the target heart rate are stored in a log that can be reviewed and/or downloaded later.

On the other hand, if the comparison step 204 determines that the user's heart rate Ra is less than the target Rt (step 220), then a second subroutine 222 may be initiated, similar to subroutine 206, but wherein increasingly intense prompts may be used to urge the user to pick up his pace and exert himself or herself harder. These will include an initial prompt 224 that serves as a first warning that Rt is not being reached. Subsequent failure to reach Rt will initiate the selection (step 226) of secondary prompts, followed by the prompt files being played (steps 228, 230, 232, 233). These intensified voice prompts occur for a pre-programmed number of times 227 before the system assumes the exerciser is no longer responding to prompts to maintain the pre-programmed regimen and an audible voice prompt would at that time state something such as “Maybe this regimen is to much for you at this time, so I'm going to just monitor your heart rate for now.” The system and method then switch to “monitor mode” 211.

In one embodiment, the following are the initial 208 and 224 voice prompts and the selectable voice prompts 210 and 226 by the system for an exerciser who is not reaching the target heart rate:
1st voice prompt response (208) “Pick up your pace”
2nd voice prompt response (212) “Come on. Try a little harder”
3rd voice prompt response (214) “You're not getting there, let's pick it up some more”
4th voice prompt response (216) “I know you can do it, push a little harder.
5th voice prompt response (217) “OK, either you're not listening to me or we set too high a goal. I'm switching to monitor mode if your pace hasn't increased in the next interval.”
6^{th} voice prompt response (224) “Slow down that pace”
7^{th} voice prompt response (228) “Come on, slow it down more”
8^{th} voice prompt response (230) “You're still not getting your pace down, relax a little”
9^{th} voice prompt response (232) “This is your fourth prompt to slow your pace. I know you can do it”
10^{th} voice prompt response (233) “This is your last chance. You're not doing what I'm asking you to do. Lower your pace or I'll stop coaching you and we'll go to monitor mode for the remainder of this regimen”

If the step 220 determines that the user is in effect meeting the target rate Rt, then the processor plays an approval prompt (step 234) in which a voice prompt file is played that informs the user that he or she is on target; for example, “That's better, nice work, now hold this pace,” or words to that effect. In one embodiment, the system prompts may be set to default to those provided from the factory or may be individually programmed and customized by the exerciser/user and downloaded to the hand held device.

Other prompts may be used depending on changes to the exerciser's performance. For example, the amount of the change in the difference between the target rate and the actual rate may be considered in selecting the prompt to play. Where the exerciser has moved closer to the target rate but is still below the target, the prompt may acknowledge the positive change with something similar to:
“You are getting closer, keep it up!”
   When the exerciser's performance is worsening, a prompt similar to the following may be given:
“You have moved even further away from your goal; try harder to change your pace.”

In the event that the exerciser remains in the category of over or under the target heart rate for five prompts, the system in one embodiment is configured to enter “monitor mode” in which the system simply announces the exerciser's heart rate at an interval. In another embodiment, the monitor mode just provides an audible voice callout of the wearer's heart rate any time the wearer pushes a selected button on the processor. A sample announcement is: “Your heart rate is 135.”

Additionally, the processor also runs a check on the actual duration 236 taken by the user to perform the selected exercise routine against a selected or programmed duration. If the duration actually taken exceeds the target duration, the processor will initiate the selection of a termination or cool down prompt 238, in which the user is informed that the exercise routine is over, and he or she can cool down to a stop. The cool down period may also have a duration that can be programmed, be a percentage of the exercise duration, be dependent on the level of target heart rate, can just be hard number such as five minutes every time, or may be selected according to some other basis.

It will be appreciated by one of skill in the art that when a comparison of a heart rate measurement Ra against a target heart rate Rt is made, some variation from exact equality may be permitted before a warning prompt is given. For example, a range or tolerance band may be used. This percentage variation from Rt may be one of the parameters that the exerciser programs into the device 12 before commencing his or her exercise routine. Typically, a user will select a narrower band of variation around Rt once he/she has used the system of the present invention a number of times, and gets a feel for how much leeway he/she should give himself/herself to obtain a satisfying exercise experience before being prompted.

Similarly, the exactly measured heart rate Ra may not be used in a comparison 204 or 220, but as is known in signal processing theory, an average or otherwise “smoothed” rate may be used to avoid excessive or spurious warnings about heart rates or unwarranted intense coaching prompts.

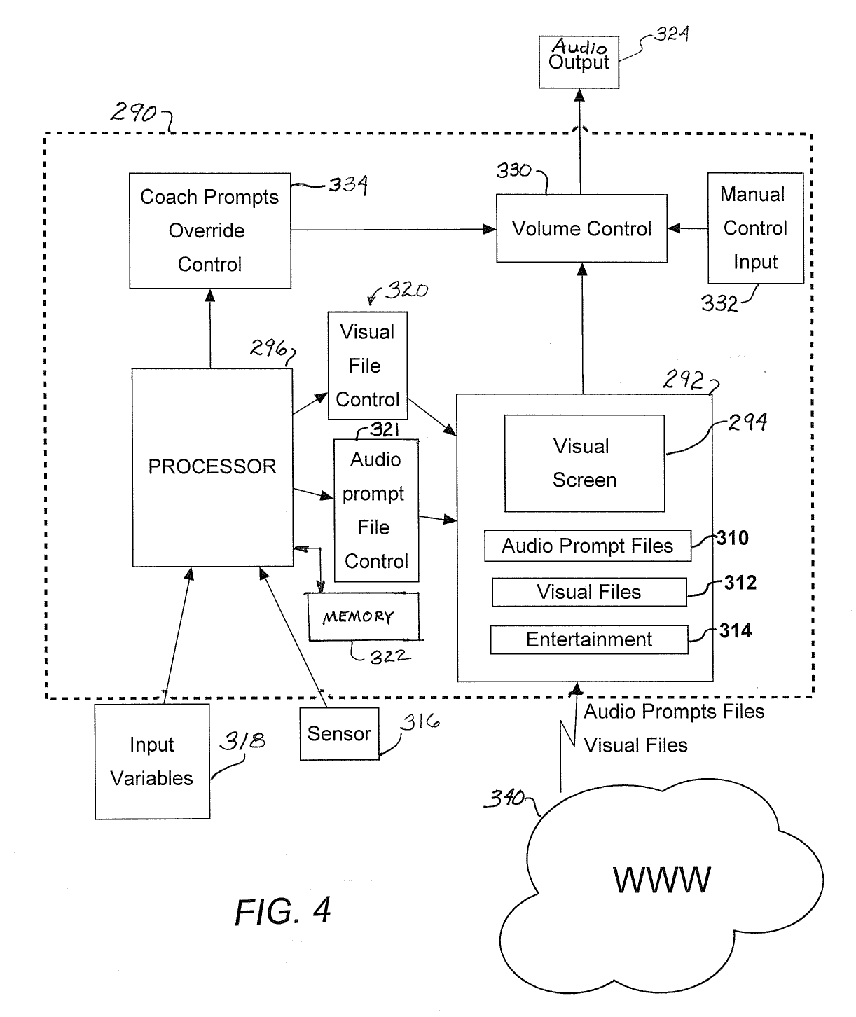

Referring now to FIG. 4, a block diagram shows an embodiment of an audible biofeedback heart rate monitor with virtual coach system 290 and certain controls in accordance with aspects of the invention. A storage and display device 292 has a display screen 294 on which images or video may be shown, or a touch pad presented, or both. A processor 296 is included within the system 290. The system is preprogrammed with audio prompt files 310 and in this embodiment, with visual files 312, which may comprise images of the face of a coach for example, or may be video files of a coach expressing prompts for example. In the case of video files, the audio may be contained therein or synchronized with a related audio file, or other approaches may be used. Other entertainment or distraction sources may be provided and are labeled “entertainment” 314 for convenience in FIG. 4. Such other sources of distraction may include live broadcast radio for example, in which case a receiver or receivers would be incorporated.

A sensor 316 feeds heart rate data to the processor 296 and the exerciser enters variable parameters by means of a keypad, keyboard, or other device 318 that will define his upcoming exercise routine and perhaps certain heart rate data processing limitations or features. The user is able to control 320 and 321 which coach will be selected, and whether it will be strictly an audio coach 310 with or without an image or a video coach 312, or which other entertainment source 314 will be used during the upcoming exercise routine. Such selections may be input to the processor 296 through the input device 318 or other input device. In the case where the exerciser has multiple coaches to choose from, such as a female coach, a male coach, a coach with a particular accent, a coach speaking a certain language, or a coach that is much more intense than another, the exerciser may make that choice at any time in one embodiment. Also, the system 290 may store such selections in a memory 322 to be used at the next exercise routine, unless overridden. In such a case, the system “remembers” what selections the exerciser made last time and automatically brings them to the exerciser's attention, asking if he or she wants to use them again. The exerciser can affirm their use or override any one of them or all of them.

After beginning the exercise routine, the audio of the coach will be output to the exerciser through an output device 324 such as earphones or a speaker, at the appropriate times as selected when the variables were input 318. In the aspect where the display and storage device 292 comprises visual images or videos of a coach, those visual images, or videos may be presented on the display screen 294 as the coach audio is played thus using the visual screen as an output. During the exercise routine if possible, the exerciser may view these visual images or videos as desired for the comfort of putting a face to a voice. Digital video files such as “.mov” files may also be played on the screen 294 as desired. In another aspect, the exerciser may change the coach in the middle of the exercise if he or she decides that a change is in order. In a further aspect, the exerciser may simply select from a list of coach names, descriptions, languages spoken, genders, and other identifying characteristics and the system 290 will load and play audio and video files that exist for that selected coach. By this system and method, a great flexibility and variety is introduced, and the exerciser is entertained at the same time as being trained. However, one of the most dynamic aspects of this invention is to provide users with the feeling of being in the company of a virtual coach, right there with them, monitoring their every aspect of exertion. Additionally, the system 290 may have an export port through which the audio and video may be output to another device, such as a television, in the case where the exerciser's routine is conducted at a single location and he or she can observe this external device during that routine. Such may be the case with a treadmill.

In a further aspect, volume control is provided so that the exerciser will be able to clearly hear the selected entertainment source and coaching prompts. A volume control 330 is provided that may be manually controlled 332 for an overall volume level per normal practice. However, in accordance with one embodiment, an automatic volume control 334 is provided that will lower the entertainment source volume when a coach's voice prompt is played via audio file. After the coaching prompt is complete, the automatic volume control 334 will then turn the entertainment volume level back up again to the exerciser-selected level.

Coaching prompt audio files, visual files, and videos may be produced and stored (310 and 312) or, as available, downloaded from other sources such as through the world-wide web (www) 340 or by other means. They may be pre-recorded by other individuals, including people with well-known voices and faces, such as sports stars or other celebrities. Actual people are used for the audio. Their voices saying the desired coaching prompts are pre-recorded and stored for use in coaching the exerciser. Actual visual images of those persons or videos of them may also be pre-recorded and stored, although in some cases, animations may be created and used.

Video and audio devices may be designed and developed or may be provided by adapting existing devices. For example, iPod™ devices, iPhone™ and other smart phones, MP3™ players of many types, PDAs (such as Palm™ PDAs), or other audio or audio/video devices may be used. Some have been miniaturized to a point where many athletes and people doing ordinary exercise may wear these lightweight devices attached to their body or clothing while exercising. For example, a user may wear one of these instruments while running, or walking These instruments may also be connected by wire to earphones that are inserted into the user's ears to deliver audio signals. Typically, such a processor may be worn on the user's wrist or upper arm or belt.

In the case of existing devices, an application program may be written encompassing FIGS. 2 and 3. Existing memory in the device may be used for the program and for stored files. In some cases, the existing operating system may be used. In other cases, modifications may be necessary.

Although shown in some cases as a wireless connection and in others as a wired connection, current technology allows for either in many cases. “Bluetooth™” technology is usable for many short distance connections and it, or equivalent wireless communication means, may be used.

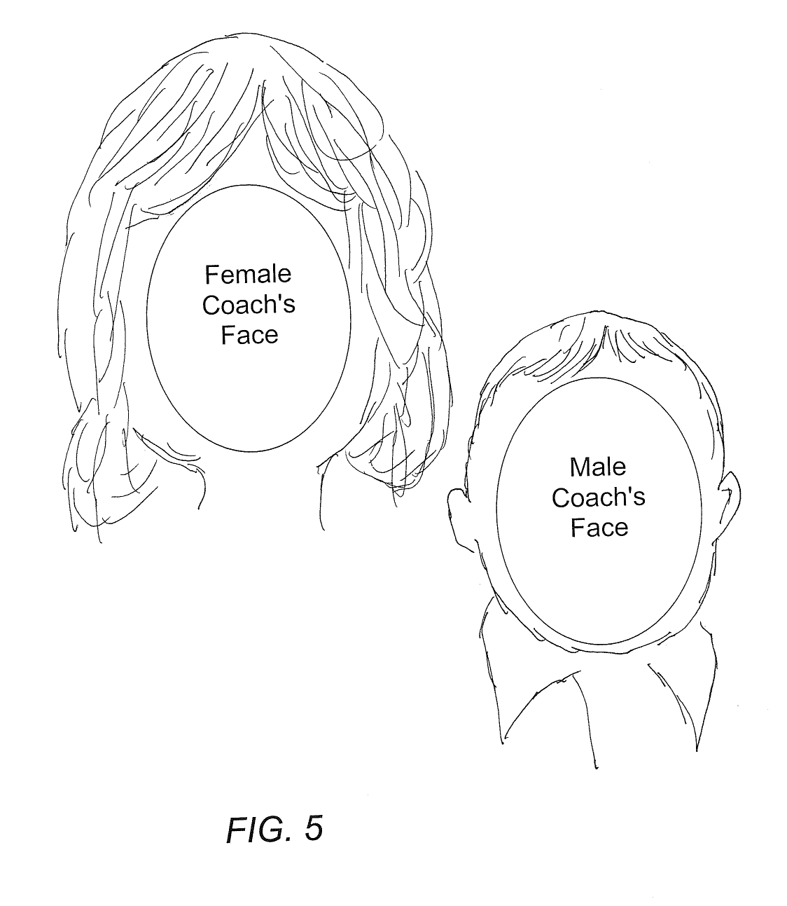

Turning now to FIG. 5, basic form diagrams of a female and a male coach's head are provided. In one aspect, these forms may be digitally processed and used as the basic gender head shapes. The processor could be programmed to overlay a selected coach's face upon the appropriate form, male or female. This selected coach head would be displayed on the visual screen 294 in FIG. 4 when a prompt is being issued by the system 290 to the exerciser. Such an approach may result in the use of less memory and processing power for providing a visual image of a coach. However, the visual image is quite basic. In another aspect, a hand-held or arm-mountable device 350 is shown in FIG. 6 with which a visual image of a coach as well as certain data can be presented on its screen 352. Earphones 354 are provided as well as certain control or input knobs or buttons 356 and input/output ports 358. Although a mini-socket connector, USB connector, and camera connector socket are shown, other connectors may be used as needed. The process of downloading video *.mov files, JPEG images and other relatively large files will benefit from USB technology or other digital connection types that provide for high speed, accurate data transfer. The actual image of a coach can be in color, black and white, depending on the image stored in the device 350. If the screen were configured differently, the coach's head may be much larger taking up most of the screen, but after the prompt is given, the coach's head may disappear and the data 362 return to the screen. Data may be displayed in different sizes with different colors or fonts, as shown in the example of FIG. 6.

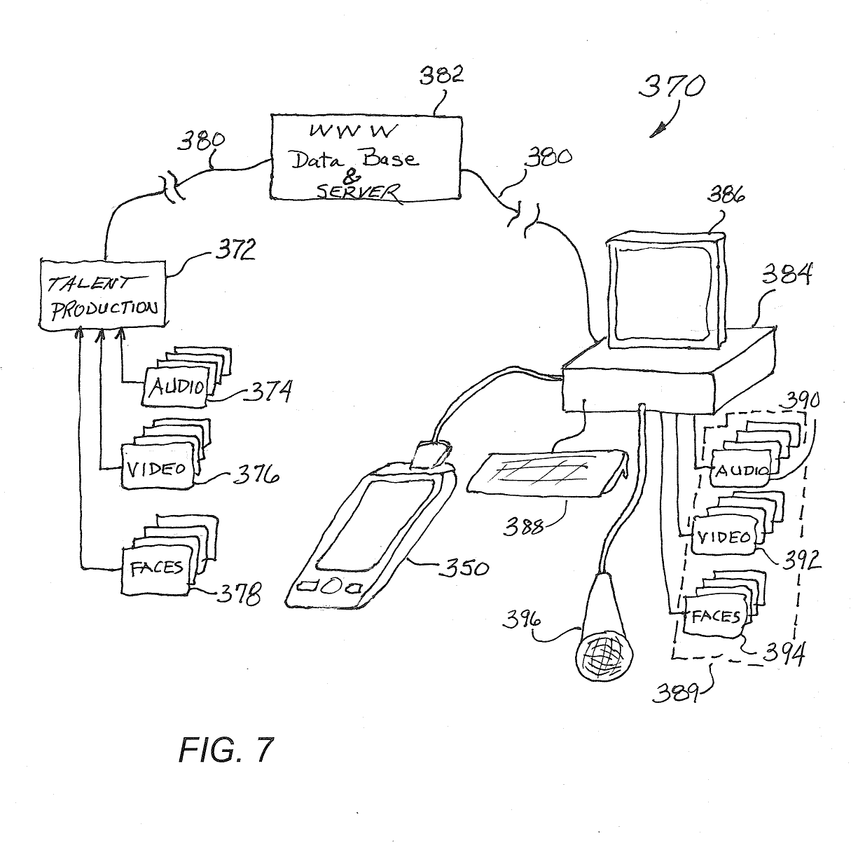

FIG. 7 presents an overall virtual coaching system 370 showing two examples of means to develop and communicate audio, video, and visual virtual coaching files to an exerciser's hand held device 350. In one approach, a talent production company 372 or other type of business records celebrity's voices 374, videos 376, and/or facial visual images 378 for use in the virtual coaching system 370. Those files are uploaded by the Internet 380 to a server and data base system 382. In this case, the server and data base system 382 are located on the world wide web (WWW) but they may be made available through other means. Likewise, communicating with the server/data base system 382 is described and shown as being through the Internet, but other means of communication may be available. In one embodiment, the server/data base system 382 takes the form of a web site capable of previewing the files, taking orders for files, charging the customer appropriate amounts for downloading the files, and actually downloading purchased files on the Internet. One such system available today is the iTunes™ system.

Once an exerciser has downloaded a virtual coaching file from the server/data base system 382, it may be stored on the exerciser's computer 384. In this case, the exerciser's computer has a display 386, keyboard 388, and memory 389 in which are stored audio files 390, video files 392, and/or visual images 394, such as faces of coaches. The memory 389 may store purchased files from the server/data base system 382, or files may be made by others, such as the exerciser himself/herself. For example, the exerciser may desire that a family member record coaching audio files for the virtual coaching system. This is easily done with almost any computer and the appropriate software today. Many computer programs are available that will digitize a speaker's voice into a useful file. Programs are available for editing that file and compressing it as needed. Audio products from Magix AG, 1105 Terminal Way #202, Reno, Nev. 89502, USA are useful for such purposes. A microphone 396 is shown connected to the computer's 384 sound card that will typically include an analog-to-digital converter controlled by the Magix™ program for creating a virtual coaching file. Likewise a camera may be connected to the computer's 384 video card for creating coaching videos.

In the iTunes™ system, the exerciser would be able to see and hear the downloaded files on the computer 384. If desired, the exerciser may download these files to the hand held device 350 on which they may be played in accordance with the embodiments discussed above. The hand held device would include a processor with the appropriate programming so that the files can be downloaded with the appropriate designations, identifications, tags, and titles for playing in accordance with embodiments discussed above. This could be done with the local iTunes™ program installed on the computer 384.

Coaching prompts may also come from other sources. For example, they may be contained on a compact disc (CD) or digital versatile disc (DVD) that is uploaded to the computer 384 through the regular means. The local iTunes™ program is capable of doing this and managing the uploaded files as discussed above.

Although discussed in general terms above, the input of parameters may be made more complex or may be made simpler as desired. For example, the heart rate data entry may enable the exerciser to input a tolerance for heart rate below the maximum rate but no tolerance above the maximum rate. That is, should the exerciser's heart rate exceed the target heart rate at any time, a coach's warning would immediately be played. However, if the exerciser's heart rate is not as high as the target heart rate, no coach's warning may be issued until the actual heart rate is some selectable percentage below the target, such as twenty-five percent. Various other specifications may be entered. The monitor mode may be overridden when an exerciser does not want the system to stop playing prompts, and those secondary prompts may then just repeat on a rotating basis, or other orders or prompts may be used. On the other hand, default settings may be programmed into the device with which the exerciser simply needs to only press a “GO” switch to engage the device and the default settings.

Thus, the present invention addresses with novel and useful features certain needs that are found in the art. The present invention may, of course, be carried out in other specific ways than those herein set forth without departing from the essential characteristics of the invention. The present embodiments and drawings are, therefore, to be considered in all respects as examples, illustrative, and not restrictive.

What is claimed is:

1. A system for monitoring a physiological parameter and coaching an exerciser during exercise, comprising:
a sensor configured to sense a physiological parameter and provide a sensor signal representative of the actual level of the sensed parameter;
an input device configured to provide a target level of the physiological parameter;
a processor programmed to receive and compare the target level of the physiological parameter with the actual sensed level and in response to the comparison, play an audible coaching prompt indicative of the difference between the actual and the target; and
an audio transducer configured to receive the played audible coaching prompt and transduce it into audio energy for listening by the exerciser.

2. The system for monitoring and coaching of claim 1 wherein the physiological parameter sensed comprises heart rate.

3. The system for monitoring and coaching of claim 1 wherein additional coaching prompts are used that vary in intensity of coaching.

4. The system for monitoring and coaching of claim 1 wherein at least one prompt comprises a recording of a voice message from an actual person.

5. The system for monitoring and coaching of claim 1 wherein the processor is further programmed to select a prompt based on whether the actual sensed parameter is higher or lower than the target rate.

6. The system for monitoring and coaching of claim 1 further comprising a memory, wherein the processor is further programmed to store in the memory the last prompt provided and in the event that a subsequent prompt is needed, the processor is further programmed to refer to the stored prompt in selecting the next prompt.

7. The system for monitoring and coaching of claim 1 further comprising a memory, wherein the memory has stored a plurality of prerecorded audio prompts from which the processor is programmed to select in playing an audio coaching prompt, the stored prompts having a predetermined sequence for playing, the processor being programmed so as to retrieve and play the prompts in the order of that predetermined sequence.

8. The system for monitoring and coaching of claim 1 further comprising a memory and a display, wherein the memory has stored a visual image of a coach face, and wherein the processor is further programmed to retrieve that visual image from the memory and output that image to the display when playing an audio prompt.

9. The system for monitoring and coaching of claim 1 further comprising a memory and a display, wherein the memory has stored a video of a coach providing the audio prompt played by the processor, the processor being further programmed to retrieve that video from the memory and output that image to the display when playing an audio prompt.

10. The system for monitoring and coaching of claim 9 further comprising a memory and an input port, wherein the processor is further programmed to receive a live video prompt at the input port, record the live prompt as a video file, and store that recorded video file in the memory for playing as a prompt.

11. The system for monitoring and coaching of claim 9 further comprising a memory and an input port, wherein the processor is further programmed to receive a recorded video prompt file at the input port and store that received video file in the memory for playing as a prompt.

12. A system for monitoring a physiological parameter and coaching an exerciser during exercise, comprising:
a sensor configured to sense heart rate and provide a sensor signal representative of the actual heart rate level;
a memory in which are stored a plurality of coaching prompts, wherein the stored coaching prompts vary in intensity of coaching and have a predetermined sequence for playing;
an input device configured to provide a target level of the heart rate;
a processor programmed to receive the input target level of the heart rate and store it in memory, the processor further programmed to compare the target level of heart rate with the actual sensed heart rate and in response to the comparison, retrieve a coaching prompt indicative of the difference between the actual and the target heart rates, the processor being programmed so as to retrieve and play the prompts in the order of that predetermined sequence and to consider any relationship between a former prompt and the present difference, from the memory and play the retrieved coaching prompt; and
a transducer configured to receive the played coaching prompt and transduce it into a perceptible communication.

13. The system for monitoring and coaching of claim 12 wherein the memory has stored a plurality of prerecorded audio prompts from which the processor is programmed to select in playing an audio coaching prompt.

14. The system for monitoring and coaching of claim 13 further comprising a display, wherein the memory has stored a visual image of a coach face, and wherein the processor is further programmed to retrieve that visual image from the memory and output that image to the display when playing an audio prompt.

15. The system for monitoring and coaching of claim 13 further comprising a display, wherein the memory has stored a video of a coach providing an audio prompt played by the processor, the processor being further programmed to retrieve that video prompt from the memory and output that image to the display while playing associated audio.

16. A method of monitoring a physiological parameter and coaching an exerciser during exercise, comprising:
measuring a physiological parameter of an exerciser;
selecting a target level for the physiological parameter of an exerciser;
comparing the measured physiological parameter to the target level;
outputting a coaching audio prompt to an exerciser based on the difference between the measured physiological parameter and the target physiological parameter; and
in the event that a visual image or a video of a coach saying an audio prompt is desired, outputting a visual image and/or a video of a coach to an exerciser.

17. The method of monitoring and coaching a person of claim 16 wherein the step of measuring comprises measuring heart rate.

18. The method of monitoring and coaching a person of claim 16 further comprising outputting additional coaching prompts that vary in intensity.

19. The method of monitoring and coaching a person of claim 16 wherein any or all of the steps of outputting a coaching audio prompt, and outputting a visual image of a coach, and outputting a video of a coach comprise using pre-recorded audio, visual, or video of real people.
